(19) United States
(12) Patent Application Publication
(10) Pub. No.: US 2026/0091193 A1
(43) Pub. Date: Apr. 2, 2026

(54) Non-Invasive Cognitive Device

(71) Applicants: Alicia Catbagan-Tapiador, New Smyrna Beach, FL (US); Arlene Rivera, Humble, TX (US)

(72) Inventors: Alicia Catbagan-Tapiador, New Smyrna Beach, FL (US); Arlene Rivera, Humble, TX (US)

(21) Appl. No.: 19/020,287

(22) Filed: Jan. 14, 2025

Related U.S. Application Data
(60) Provisional application No. 63/699,845, filed on Sep. 27, 2024.

(51) Int. Cl. A61M 21/00

(52) U.S. Cl. CPC A61M 21/00; A61M 2021/0027

(57) ABSTRACT

A non-invasive cognitive device is disclosed, which produces an electrical stimulation which mimics the strength of energy produced in the brain. The non-invasive cognitive device comprises a small electromagnetic disc and monitoring system that transmits signals to the brain. The disc is attached to an anchor and positioned behind a user's right ear. The disc, which is paired to a monitoring system, sends electromagnetic signals with music directly to a user's brain. Nurses or caregivers can monitor which music positively or negatively affects the user's state of mind. Specifically, users transmit electrical stimulation and music to the right temporal lobe which is then communicated to the left temporal lobe. This is then transmitted to the medulla oblongata to ease or calm down the uncontrolled behavior associated with dementia, depression, Alzheimer's, anxiety, etc.

### CROSS-REFERENCE TO RELATED APPLICATION

The present application claims priority to, and the benefit of, U.S. Provisional Application No. 63/699,845, which was filed on Sep. 27, 2024, and is incorporated herein by reference in its entirety.

### FIELD OF THE INVENTION

The present invention relates generally to the field of non-invasive cognitive devices. More specifically, the present invention relates to a three-part device that delivers electromagnetic waves on a low resting voltage to help with conditions like dementia, depression, anxiety, and more. Accordingly, this disclosure makes specific reference thereto the present invention. Nonetheless, it is to be appreciated that aspects of the present invention are also equally applicable to other like applications, devices, and methods of manufacture.

### BACKGROUND

By way of background, this invention relates to improvements in non-invasive cognitive devices. Generally, patients with cognitive impairments may require several treatments to help alleviate symptoms and ailments associated with their condition. Without treatment, patient confusion will progress faster and lead to behavioral issues like anxiety, restlessness, etc. Further, patient quality of life can deteriorate and impact daily activities like communicating with others, using the bathroom independently, and more. Additionally, dependence on psychotropic medications can increase over time and lead to deteriorated treatment effectiveness.

Millions of individuals suffer from clinically diagnosed anxiety on a daily basis. Countless other people suffer from dementia and/or depression, etc. While there are various medications to treat these conditions, there are no reliable, widely accepted non-pharmaceutical treatment options available to sufferers. It has been postulated that if an effective means existed for controlling and mimicking the strength of energy produced in the brain, patients suffering from abnormal brain conditions could have improved quality of life through a non-invasive means. In view of this, the disclosed invention provides a variety of brain stimulation methods, such as sending electromagnetic signals with music directly to the medulla oblongata to potentially reduce erratic behavior caused by identified behaviors associated with dementia, depression and anxiety, etc.

Accordingly, there is a demand for an improved non-invasive cognitive device that allows users to deliver electromagnetic waves on a low resting voltage to help with conditions, like dementia, depression, anxiety, etc. More particularly, there is a demand for a non-invasive cognitive device that sends electromagnetic signals with music directly to the medulla oblongata to potentially reduce erratic behavior.

Therefore, there exists a long-felt need in the art for a non-invasive cognitive device that provides users with a three-part device that delivers electromagnetic waves on a low resting voltage to help with conditions like dementia, depression, anxiety, and more. There is also a long-felt need in the art for a non-invasive cognitive device that improves quality of life for patients suffering from abnormal brain conditions through non-invasive and non-pharmacological intervention. Further, there is a long-felt need in the art for a non-invasive cognitive device that features a tiny electromagnetic disk that is paired with a monitoring system that sends electromagnetic signals with music directly to the medulla oblongata. Moreover, there is a long-felt need in the art for a device that potentially reduces erratic behavior caused by identified behaviors associated with dementia, early onset Alzheimer's, and major depression. Further, there is a long-felt need in the art for a non-invasive cognitive device that mimics the strength of energy produced in the brain. Finally, there is a long-felt need in the art for a non-invasive cognitive device that simultaneously transmits electrical stimulation and music to the right temporal lobe and connects information to the left temporal lobe.

The subject matter disclosed and claimed herein, in one embodiment thereof, comprises a non-invasive cognitive device. The device produces an electrical stimulation which mimics the strength of energy produced in the brain. The non-invasive cognitive device comprises a small electromagnetic disc and monitoring system that transmits signals to the brain. The disc is attached to an anchor and positioned behind a user's right ear. The disc, which is paired to a monitoring system, sends electromagnetic signals with music directly to a user's brain. Nurses or caregivers can monitor which music positively or negatively affects the user's state of mind. Specifically, users transmit electrical stimulation and music to the right temporal lobe which is then communicated to the left temporal lobe. This is then transmitted to the medulla oblongata to ease or calm down the uncontrolled behavior associated with dementia, depression, Alzheimer's, anxiety, etc.

In this manner, the non-invasive cognitive device of the present invention accomplishes all of the foregoing objectives and provides users with a device that stimulates the brain to reduce erratic behavior. The device is an electromagnetic disc and monitoring system positioned behind a user's ear. The device transmits electrical stimulation and music.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a non-invasive cognitive device. The device produces an electrical stimulation which mimics the strength of energy produced in the brain. The non-invasive cognitive device comprises a small electromagnetic disc, earphone, and monitoring system that transmits signals to the brain. The disc and earphone are positioned near a user's ear. The disc and earphone are paired to a monitoring system, which sends electromagnetic signals with music directly to a user's brain to ease or calm down the uncontrolled behavior associated with dementia, depression, Alzheimer's, anxiety, etc.

In one embodiment, the present invention seeks to address and resolve the problems experienced by individuals suffering from uncontrolled anxiety associated with depression, dementia, and Alzheimer's, etc., or any other suitable condition which might produce involuntary elevated stress levels within a user and cause the user to exhibit erratic behavior. Embodiments of the present invention allow users to discretely control their erratic behavior and potentially reduce psychotropic medication usage to improve the quality of life of the patient.

In one embodiment, the non-invasive cognitive device comprises an electromagnetic disc. The electromagnetic disc includes a transmitter/receiver and any other circuitry needed which receives signals and transmits signals to the brain. Specifically, the electromagnetic disc transmits electromagnetic waves on a low resting voltage to a user's brain. The electromagnetic disc is approximately the size of a penny, i.e., approximately 19.05 mm and attached on an adhesive patch, and applied behind a user's right ear. Typically, the electromagnetic disc is circular or oval in shape and sized to be positioned behind a user's right ear.

In one embodiment, the electromagnetic disc is hardwired with an anchor and secured. The anchor would have an adhesive patch which would allow a user to stick the disc behind a user's right ear and remove it as needed. Specifically, the disc and anchor include an adhesive patch, such that the user can choose to place the disc directly on their body. With this configuration, the user can be extremely specific about where they want to place the disc. Thus, the individual is able to place and maintain the electromagnetic disc firmly in contact with his or her skull with various orientations through the anchor. Specifically, the electromagnetic disc can be placed behind the right ear at the base of the skull to allow the electric signals to transmit directly through bone conduction. The electromagnetic disc can also be placed on other parts of the skull as long as the periodic electromagnetic waves are being properly imparted to and decoded by the user's brain. When using electromagnetic waves and with the disc placed behind the right ear (or on the skull), the signals bypass the eardrum and are imparted directly to the cochlea.

In one embodiment, the non-invasive cognitive device also comprises an earphone or earbud and can be secured within a headset. The headset is hardwired to an anchor and a flexible connector is attached to the disc with an adhesive patch. The earphone can be any suitable shape and size as is known in the art, as long as the earphone is sized and shaped to fit within a user's ear. Further, the earphone includes a transmitter/receiver and any other circuitry needed which receives signals and transmits music into the user's ear. Specifically, the earphone transmits music to a user's brain simultaneously as the electromagnetic disc transmits the electric signals to a user's brain. Typically, the music used is a combination of music of their choice produced the year the patient was born. Generally, nurses or caregivers can monitor which music positively or negatively affects the user's state of mind.

In one embodiment, the electromagnetic disc and the earphone are paired with and in communication with a monitoring system. The monitoring system includes a transmitter/receiver and any other circuitry needed within a housing and a frequency transmitting element/transducer. Further, additional components used to operate and monitor the non-invasive cognitive device include a printed circuit board (“PCB”) that houses a microcontroller and associated memory on the PCB. The memory is communicatively connected to, or embedded within the microcontroller, and to the transmitter/receiver or transceiver components. In a primary embodiment of the device, the user places the disc behind his or her right ear, at the base of the skull. A user can then activate the device when they are experiencing erratic behavior or anxiety and stress, or whenever they feel the need for an immediate clear mind. The individual simply places the disc at or on the instructed location (i.e., right ear), turns the device on, and his or her brain state will quickly react to the simultaneous transmission of music and electromagnetic waves via control by the monitoring system which is in communication with the disc and earphone. This system involves another person to observe the patient, to ensure that anxiety de-escalates, calms down slowly during the treatment.

In one embodiment, all the components of the device are extremely compact and portable for easy transportation and storage. Such a compact configuration allows users to operate and use the device discreetly. Oftentimes, anxiety attacks, depression, and intense episodes of stress do not occur in the comfort of an individual's home, but rather at work, public or social events, and other various settings where the individual is not alone and comfortable. An individual will be able to readily carry the device in their pocket or any type of bag. This allows users to address the need to counter anxiety and depression, as well as de-escalate stress wherever they may be.

In one embodiment, the electromagnetic disc and the earphone are housed within a headset. The headset includes the disc hardwired in and when manipulated, the disc will be able to reach the back of the user's ear for placement there. This will also be connected to and in communication with the monitoring system. Typically, the disc and the earphone are positioned within the right headphone of the headset.

In one embodiment, the electrical stimulation provided (i.e., the electromagnetic waves and the music) mimics the strength of energy produced in the brain. Using the two-way module, users can simultaneously transmit electrical stimulation and music to the right temporal lobe and communicates information with the left temporal lobe. This information is then transmitted/communicated with the medulla oblongata to ease or calm down the uncontrolled behavior associated with dementia, depression, anxiety, Alzheimer's, etc.

In one embodiment, the monitoring system can be in communication with a smart device, which records various physiological and device metrics, as well as related usage data, as such data is collected through a wireless or wired system that communicates between the user's smartphone, tablet or device, a database, and the non-invasive cognitive device.

In a further embodiment, the device may also include a transmitter/receiver component capable of transmitting data gathered by the device to a remote database. This transmitter/receiver component will record when the device is turned on, the duration of use, as well as other metrics or factors to record trends and patterns. The user will be able to download an external application on a smartphone, or tablet or visit a website to view the recorded data and metrics.

In one embodiment, the device can transmit or transmit and receive the recorded data through a variety of means. In one embodiment, the device records the data and sends it to the user's phone, tablet, or other remote device via Bluetooth or similar communication technology. The data is then sent to a database from the phone where it is stored for later access or analysis. Further, the user's phone, tablet, or other device can receive relevant data from the device, and/or sends relevant data to the device through known two-way signal communication protocols, such as Wi-Fi or Bluetooth protocols. The database may also be communicatively connected with the user's phone to allow the user to view the trends and data in a user-friendly fashion. In further embodiments, the device may be configured in other architectures with additional communication components that alternatively allow the data to be recorded and stored, and later displayed to the user.

In one embodiment, clinical professionals such as psychologists and psychiatrists, as well as parents, caretakers, and other authorized recipients may also be able to download the application to view the user's data. Clinical professionals or caretakers can then review the data to determine any trends or other valuable data to gain a better insight on the user's condition and then may be able to structure more personalized treatment, coping methods, and focused therapy sessions.

In one embodiment, the device is powered by a rechargeable and/or a removable battery and may have a recharging port on the monitoring system.

In operation, the electromagnetic disc of the non-invasive cognitive device is positioned behind a user's ear (designated person), typically the right ear. The earphone is then positioned within the user's ear. Both the electromagnetic disc and the earphone are in communication with a monitoring system. The user then monitors and controls the simultaneous transmission of electromagnetic waves and music to the brain. Specifically, the electromagnetic waves and the music is transmitted to the right temporal lobe and communicated to the left temporal lobe, then transmitted to the medulla oblongata to potentially reduce erratic behavior caused by dementia, depression, anxiety, Alzheimer's, etc., and to gradually reduce psychotropic medication usage to improve the quality of life of the user.

In yet another embodiment, the non-invasive cognitive device comprises a plurality of indicia.

In yet another embodiment, a method of providing electrical stimulation to the brain to reduce erratic behavior is disclosed. The method includes the steps of providing a non-invasive cognitive device comprising an electromagnetic disc and an earphone that are paired with a monitoring system. The method also comprises positioning the electromagnetic disc behind a user's ear. Further, the method comprises positioning the earphone into the right ear. The method also comprises transmitting low frequency electrical impulses and music to the user via the electromagnetic disc and earphone. Finally, the method comprises monitoring the user's reaction to the music and the user's actions to ease erratic behavior.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the non-invasive cognitive device of the present invention showing the electromagnetic disc and monitoring system in accordance with the disclosed architecture;

FIG. 2 illustrates a perspective view of one embodiment of the non-invasive cognitive device of the present invention showing the device in position behind a user's ear in accordance with the disclosed architecture;

FIG. 3 illustrates a perspective view of one embodiment of the non-invasive cognitive device of the present invention showing the part of the brain the device is used to stimulate in accordance with the disclosed architecture;

FIG. 4 illustrates a perspective view of one embodiment of the non-invasive cognitive device of the present invention showing the device implemented as a headset in accordance with the disclosed architecture; and

FIG. 5 illustrates a flowchart showing the method of providing electrical stimulation to the brain to reduce erratic behavior in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a non-invasive cognitive device that provides users with a three-part device that delivers electromagnetic waves on a low resting voltage to help with conditions like dementia, depression, anxiety, and more. There is also a long-felt need in the art for a non-invasive cognitive device that improves quality of life for patients suffering from abnormal brain conditions through non-invasive and non-pharmacological intervention. Further, there is a long-felt need in the art for a non-invasive cognitive device that features a tiny electromagnetic disk that is paired with a monitoring system that sends electromagnetic signals with music directly to the medulla oblongata. Moreover, there is a long-felt need in the art for a device that potentially reduces erratic behavior caused by identified behaviors associated with dementia, early onset Alzheimer's, and major depression. Further, there is a long-felt need in the art for a non-invasive cognitive device that mimics the strength of energy produced in the brain. Finally, there is a long-felt need in the art for a non-invasive cognitive device that simultaneously transmits electrical stimulation and music to the right temporal lobe and connects information to the left temporal lobe.

The present invention, in one exemplary embodiment, is a novel non-invasive cognitive device. The device produces an electrical stimulation which mimics the strength of energy produced in the brain. The non-invasive cognitive device comprises a small electromagnetic disc and monitoring system that transmits signals to the brain. The disc is attached to an anchor and positioned behind a user's ear. The disc, which is paired to a monitoring system, sends electromagnetic signals with music directly to a user's brain. Nurses or caregivers can monitor which music positively or negatively affects the user's state of mind. Specifically, users transmit electrical stimulation and music to the right temporal lobe which is then communicated to the left temporal lobe. This is then transmitted to the medulla oblongata to ease or calm down the uncontrolled behavior associated with dementia, depression, Alzheimer's, anxiety, etc. The present invention also includes a novel method of providing electrical stimulation to the brain to reduce erratic behavior. The method includes the steps of providing a non-invasive cognitive device comprising an electromagnetic disc and an earphone that are paired with a monitoring system. The method also comprises positioning the electromagnetic disc behind a user's ear. Further, the method comprises positioning the earphone into the right ear. The method also comprises transmitting low frequency electrical impulses and music to the user via the electromagnetic disc and earphone. Finally, the method comprises monitoring the user's reaction to the music and the user's actions to ease erratic behavior.

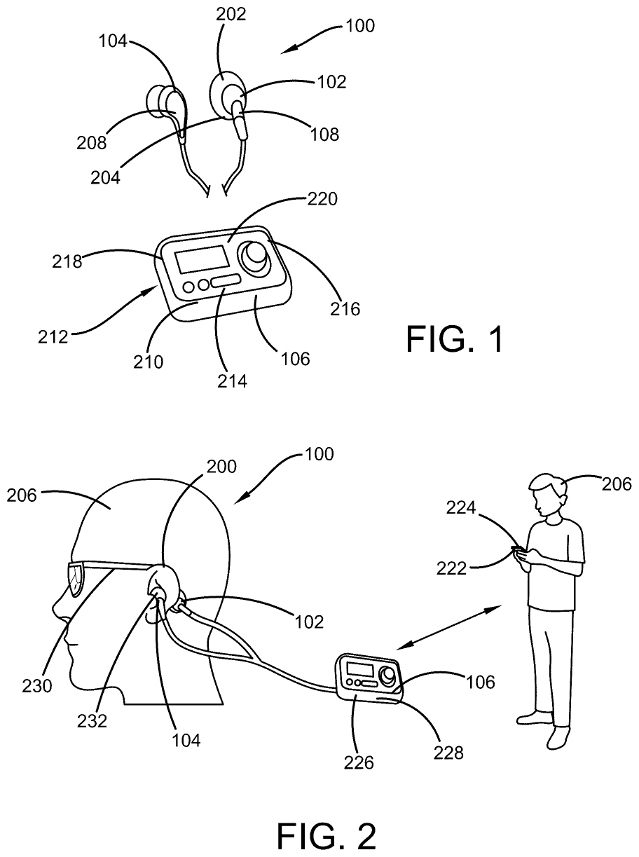

Referring initially to the drawings, FIG. 1 illustrates a perspective view of one embodiment of the non-invasive cognitive device 100 of the present invention. In the present embodiment, the non-invasive cognitive device 100 is an improved non-invasive cognitive device 100 that provides a user with a means for reducing erratic behavior caused by conditions such as, depression, anxiety, dementia, Alzheimer's, etc. Specifically, the non-invasive cognitive device 100 comprises an electromagnetic disc 102 and earphone 104 which are paired with a monitoring system 106.

Generally, the present invention seeks to address and resolve the problems experienced by individuals suffering from involuntary anxiety, depression, dementia, and Alzheimer's, etc., or any other suitable condition which might produce involuntary elevated stress levels within a user and cause the user to exhibit erratic behavior. Embodiments of the present invention allow users to discretely control their erratic behavior and potentially reduce psychotropic medication usage to improve the quality of life of the patient.

As shown in FIG. 2, the non-invasive cognitive device 100 comprises an electromagnetic disc 102. The electromagnetic disc 102 includes a transmitter/receiver 108 and any other circuitry needed which receives signals and transmits signals to the brain 300. Specifically, the electromagnetic disc 102 transmits electromagnetic waves on a low resting voltage to a user's brain 300. The electromagnetic disc 102 is typically the size of a penny, i.e., approximately 19.05 mm and attached to an adhesive patch 204. Typically, the electromagnetic disc 102 is circular or oval in shape and sized to be positioned behind a user's right ear 200.

Further, the electromagnetic disc 102 is paired with an anchor 202 and secured. The anchor 202 would have an adhesive patch 204 which would allow a user 206 to stick the disc 102 behind a user's right ear 200 and remove it as needed. Specifically, the disc 102 and anchor 202 include an adhesive patch 204, such that the user 206 can choose to place the disc 102 directly on their body. With this configuration, the user 206 can be extremely specific about where they want to place the disc 102. Thus, the individual 206 is able to place and maintain the electromagnetic disc 102 firmly in contact with his or her skull with various orientations through the anchor 202. Specifically, the electromagnetic disc 102 can be placed behind the right ear 200 at the base of the skull to allow the electric signals to transmit directly through bone conduction. The electromagnetic disc 102 can also be placed on other parts of the skull as long as the periodic electromagnetic waves are being properly imparted to and decoded by the user's brain. When using electromagnetic waves and with the disc 102 placed behind the right ear 200 (or on the skull), the signals bypass the eardrum and are imparted directly to the cochlea.

Additionally, the non-invasive cognitive device 100 also comprises an earphone 104 or earbud. The earphone 104 can be any suitable shape and size as is known in the art, as long as the earphone 104 is sized and shaped to fit within a user's ear 200. Further, the earphone 104 includes a transmitter/receiver 208 and any other circuitry needed which receives signals and transmits music into the user's ear 200. Specifically, the earphone 104 transmits music to a user's brain 300 simultaneously as the electromagnetic disc 102 transmits the electric signals to a user's brain 300. Typically, the music used is a combination of music of their choice produced the year the patient was born. Generally, nurses or caregivers can monitor which music positively or negatively affects the user's state of mind. Typically, the treatment is 15-20 minutes long, as needed.

Furthermore, the electromagnetic disc 102 and the earphone 104 are paired with and in communication with a monitoring system 106. The monitoring system 106 includes a transmitter/receiver 210 and any other circuitry needed within a housing 212 and a frequency transmitting element/transducer 214. Further, additional components used to operate and monitor the non-invasive cognitive device 100 include a printed circuit board (“PCB”) 216 that houses a microcontroller 218 and associated memory 220 on the PCB 216. The memory 220 is communicatively connected to, or embedded within the microcontroller 218, and to the transmitter/receiver 210 or transceiver components 214. In a primary embodiment of the device 100, the user 206 places the disc 102 behind his or her right ear 200, at the base of the skull. A user 206 can then activate the device 100 when they are experiencing erratic behavior or anxiety and stress, or whenever they feel the need for an immediate clear mind. The individual 206 simply places the disc 102 at or on the instructed location, turns the device 100 on, and his or her brain state will quickly react to the simultaneous transmission of music and electromagnetic waves via control by the monitoring system 106 which is in communication with the disc 102 and earphone 104.

In one embodiment, the monitoring system 106 can be in communication with a smart device 222, which records various physiological and device metrics, as well as related usage data, as such data is collected through a wireless or wired system that communicates between the user's smartphone 222, tablet or device, a database and the non-invasive cognitive device 100.

In a further embodiment, the device 100 may also include a transmitter/receiver component 108, 208, 210 capable of transmitting data gathered by the device 100 to a remote database. This transmitter/receiver component 108, 208, 210 will record when the device 100 is turned on, the duration of use, as well as other metrics or factors to record trends and patterns. The user 206 will be able to download an external application 224 on a smartphone 222, or tablet or visit a website to view the recorded data and metrics.

Further, the device 100 can transmit, or transmit and receive the recorded data through a variety of means. In one embodiment, the device 100 records the data and sends it to the user's phone 222, tablet, or other remote device via Bluetooth or similar communication technology. The data is then sent to a database from the phone 222 where it is stored for later access or analysis. Further, the user's phone 222, tablet, or other device can receive relevant data from the device 100, and/or sends relevant data to the device 100 through known two-way signal communication protocols, such as Wi-Fi or Bluetooth protocols. The database may also be communicatively connected with the user's phone 222 to allow the user 206 to view the trends and data in a user-friendly fashion. In further embodiments, the device 100 may be configured in other architectures with additional communication components that alternatively allow the data to be recorded and stored, and later displayed to the user 206.

In one embodiment, the device 100 is powered by a rechargeable and/or a removable battery 226 and may have a recharging port 228 on the monitoring system 106. The battery 226 may be a disposable battery or a rechargeable battery in the form of an alkaline, nickel-cadmium, nickel-metal hydride battery, etc., such as any 3V-12 volts DC battery or other conventional battery, such as A, AA, AAA, etc., that supplies power to the non-invasive cognitive device 100. Throughout this specification, the term “battery” may be used interchangeably to refer to one or more wet or dry cells or batteries of cells in which chemical energy is converted into electricity and used as a source of DC power. References to recharging or replacing the battery 226 may refer to recharging or replacing individual cells, individual batteries of cells, or a package of multiple battery cells as is appropriate for any given battery technology that may be used. In addition, a rechargeable embodiment of the battery 226 may be recharged using a USB port 228, wherein the USB port 228 is a USB-A, USB-B, Micro-B, Micro-USB, Mini-USB, or USB-C port, etc.

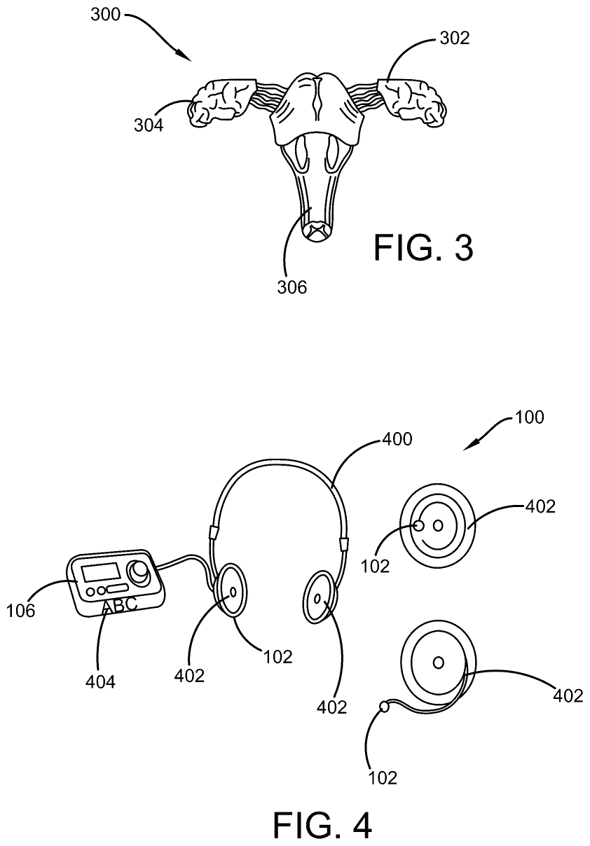

As shown in FIG. 3, the electrical stimulation provided (i.e., the electromagnetic waves and the music) mimics the strength of energy produced in the brain 300. Using the two-way module, users 206 can simultaneously transmit electrical stimulation and music to the right temporal lobe 302 and communicates information with the left temporal lobe 304. This information is then transmitted/communicated with the medulla oblongata 306 to ease or calm down the uncontrolled behavior associated with dementia, depression, anxiety, Alzheimer's, etc.

In one embodiment, clinical professionals such as psychologists and psychiatrists, as well as parents, caregivers, and other authorized recipients may also be able to download the mobile/software application 224 to view the user's data. Clinical professionals or caregivers can then review the data to determine any trends or other valuable data to gain a better insight on the user's condition, and then may be able to structure more personalized treatment, coping methods, and focused therapy sessions.

As shown in FIG. 4, all the components of the device 100 are extremely compact and portable for easy transportation and storage. Such a compact configuration allows users 206 to operate and use the device 100 discreetly. Oftentimes, anxiety attacks, depression, and intense episodes of stress do not occur in the comfort of an individual's home, but rather at work, public or social events, and other various settings where the individual is not alone and comfortable. An individual will be able to readily carry the device 100 in their pocket or any type of bag. This allows users 206 to address the need to counter anxiety and depression, as well as de-escalate stress wherever they may be.

In one embodiment, the electromagnetic disc 102 and the earphone 104 are housed within a headset 400. The headset 400 includes the disc 102 wired in and when manipulated, the disc 102 will be able to reach behind the user's right ear 200 for placement there. This will also be connected to and in communication with the monitoring system 106. Typically, the disc 102 and the earphone 104 are positioned within the right headphone 402 of the headset 400.

In operation, the electromagnetic disc 102 of the non-invasive cognitive device 100 is positioned behind a user's ear 200, typically the right ear 200. The earphone 104 is then positioned within the user's right ear 200. Both the electromagnetic disc 102 and the earphone 104 are in communication with a monitoring system 106. The user 206 then monitors and controls the simultaneous transmission of electromagnetic waves and music to the brain 300. Specifically, the electromagnetic waves and the music is transmitted to the right temporal lobe 302 and communicated to the left temporal lobe 304, then transmitted to the medulla oblongata 306 to potentially reduce erratic behavior caused by dementia, depression, anxiety, Alzheimer's, etc., and to gradually reduce psychotropic medication usage to improve the quality of life of the user 206.

FIG. 4 In yet another embodiment, the non-invasive cognitive device 100 comprises a plurality of indicia 404. The monitoring system 106 of the device 100 may include advertising, a trademark, or other letters, designs, or characters, printed, painted, stamped, or integrated into the monitoring system 106, or any other indicia 404 as is known in the art. Specifically, any suitable indicia 404 as is known in the art can be included, such as, but not limited to, patterns, logos, emblems, images, symbols, designs, letters, words, characters, animals, advertisements, brands, etc., that may or may not be brain, electromagnetic disc, or brand related.

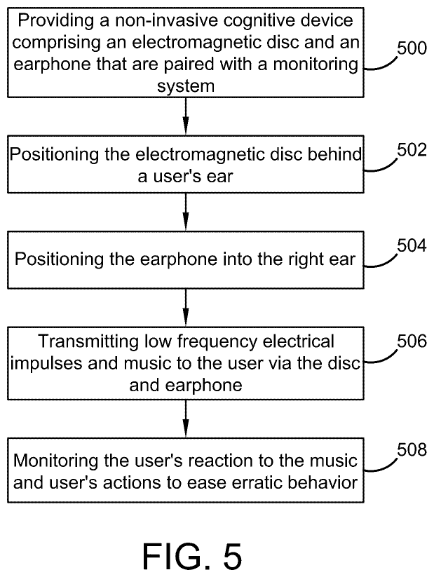

FIG. 5 illustrates a flowchart of the method of providing electrical stimulation to the brain to reduce erratic behavior. The method includes the steps of at 500, providing a non-invasive cognitive device comprising an electromagnetic disc and an earphone that are paired with a monitoring system. The method also comprises at 502, positioning the electromagnetic disc behind a user's ear. Further, the method comprises at 504, positioning the earphone into the right ear. The method also comprises at 506, transmitting low frequency electrical impulses and music to the user via the electromagnetic disc and earphone. Finally, the method comprises at 508, monitoring the user's reaction to the music and the user's actions to ease erratic behavior.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “non-invasive cognitive device”, “cognitive device”, “non-invasive device”, and “device” are interchangeable and refer to the non-invasive cognitive device 100 of the present invention.

Notwithstanding the foregoing, the non-invasive cognitive device 100 of the present invention can be of any suitable size and configuration as is known in the art without affecting the overall concept of the invention, provided that it accomplishes the above-stated objectives. One of ordinary skill in the art will appreciate that the non-invasive cognitive device 100 as shown in FIGS. 1-5 is for illustrative purposes only, and that many other sizes and shapes of the non-invasive cognitive device 100 are well within the scope of the present disclosure. Although the dimensions of the non-invasive cognitive device 100 are important design parameters for user convenience, the non-invasive cognitive device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A non-invasive cognitive device that provides a user with a means for reducing erratic behavior, the non-invasive cognitive device comprising:
an electromagnetic disc;
an earphone; and
a monitoring system;
wherein the electromagnetic disc is positioned behind a user's ear;
wherein the earphone is positioned within a user's ear;
wherein the electromagnetic disc and the earphone are paired with the monitoring system; and
further wherein the electromagnetic disc transmits electromagnetic waves and the earphone transmits music simultaneously to a user to discretely control a user's erratic behavior.

2. The non-invasive cognitive device of claim 1, wherein the electromagnetic disc includes a transmitter/receiver which receives signals and transmits electromagnetic signals to a user's brain.

3. The non-invasive cognitive device of claim 2, wherein the electromagnetic disc is paired with an anchor and secured behind a user's right ear.

4. The non-invasive cognitive device of claim 3, wherein the anchor comprises an adhesive patch to stick the electromagnetic disc behind a user's right ear and remove it as needed.

5. The non-invasive cognitive device of claim 4, wherein the earphone includes a transmitter/receiver which receives signals and transmits music into the user's right ear.

6. The non-invasive cognitive device of claim 5, wherein nurses or caregivers can monitor which music positively or negatively affects a user's state of mind.

7. The non-invasive cognitive device of claim 6, wherein the monitoring system includes a transmitter/receiver within a housing, along with a printed circuit board, a microcontroller, and a memory used to operate and monitor the non-invasive cognitive device.

8. The non-invasive cognitive device of claim 7, wherein the monitoring system is in communication with a smart device, which records various physiological and device metrics, as well as related usage data, as such data is collected through a wireless or wired system that communicates between the smart device, a database and the non-invasive cognitive device.

9. The non-invasive cognitive device of claim 8, wherein a user will be able to download an external application on the smart device or visit a website to view recorded data and metrics.

10. The non-invasive cognitive device of claim 9, wherein the non-invasive cognitive device is powered by a rechargeable battery.

11. The non-invasive cognitive device of claim 10, wherein the non-invasive cognitive device comprises a recharging port to charge the rechargeable battery.

12. The non-invasive cognitive device of claim 11, wherein the non-invasive cognitive device simultaneously transmits electrical stimulation and music to a right temporal lobe and communicates information with a left temporal lobe, then it is communicated with the medulla oblongata.

13. A non-invasive cognitive device that provides a user with a means for reducing erratic behavior, the non-invasive cognitive device comprising:
an electromagnetic disc which includes a transmitter/receiver which receives signals and transmits electromagnetic signals to a user's brain;
an earphone which includes a transmitter/receiver which receives signals and transmits music into the user's right ear; and
a monitoring system which includes a transmitter/receiver within a housing, along with a printed circuit board, a microcontroller, and a memory which is used to operate and monitor the non-invasive cognitive device;
wherein the electromagnetic disc is paired with an anchor and secured behind a user's right ear;
wherein the earphone is positioned within a user's ear and nurses or caregivers can monitor which music positively or negatively affects a user's state of mind;
wherein the electromagnetic disc and the earphone are paired with the monitoring system;
wherein the electromagnetic disc transmits electromagnetic waves and the earphone transmits music simultaneously to a user to discretely control a user's erratic behavior;
wherein the non-invasive cognitive device simultaneously transmits electrical stimulation and music to a right temporal lobe and communicates information with a left temporal lobe, then it is communicated with the medulla oblongata;
wherein the non-invasive cognitive device is powered by a rechargeable battery; and
further wherein the non-invasive cognitive device comprises a recharging port to charge the rechargeable battery.

14. The non-invasive cognitive device of claim 13, wherein the monitoring system is in communication with a smart device, which records various physiological and device metrics, as well as related usage data, as such data is collected through a wireless or wired system that communicates between the smart device, a database and the non-invasive cognitive device.

15. The non-invasive cognitive device of claim 14, wherein clinical professionals such as psychologists and psychiatrists, as well as parents, caregivers, and other authorized recipients may also be able to download and view the user's data.

16. The non-invasive cognitive device of claim 13, wherein the electromagnetic disc is approximately 19.05 mm in size.

17. The non-invasive cognitive device of claim 13, wherein the electromagnetic disc and the earphone are housed within a headset.

18. The non-invasive cognitive device of claim 13, wherein the headset includes the electromagnetic disc hardwired in and when manipulated, the electromagnetic disc will be able to reach a back of a user's right ear for placement there.

19. The non-invasive cognitive device of claim 13 further comprises a plurality of indicia.

20. A method of providing electrical stimulation to the brain to reduce erratic behavior, the method comprising the following steps:
providing a non-invasive cognitive device comprising an electromagnetic disc and an earphone that are paired with a monitoring system;
positioning the electromagnetic disc behind a user's ear;
positioning the earphone into the right ear;
transmitting low frequency electrical impulses and music to the user via the electromagnetic disc and earphone; and
monitoring the user's reaction to the music and the user's actions to ease erratic behavior.
